(19) United States
(12) Patent Application Publication
(10) Pub. No.: US 2025/0051725 A1
(43) Pub. Date: Feb. 13, 2025

(54) GENERATION OF MATRIX-BOUND NANOVESICLES FROM HUMAN BRAIN ORGANOIDS

(71) Applicant: FLORIDA STATE UNIVERSITY RESEARCH FOUNDATION, INC., Tallahassee, FL (US)

(72) Inventors: CHANG LIU, TALLAHASSEE, FL (US); YAN LI, TALLAHASSEE, FL (US)

(21) Appl. No.: 18/738,449

(22) Filed: Jun. 10, 2024

Related U.S. Application Data
(60) Provisional application No. 63/531,823, filed on Aug. 10, 2023.

(51) Int. Cl. C12N 5/0793

(52) U.S. Cl. CPC C12N 5/0619; C12N 2501/115; C12N 2501/155; C12N 2501/16; C12N 2501/21; C12N 2501/41; C12N 2501/999; C12N 2506/45; C12N 2513/00

(57) ABSTRACT

The subject invention pertains to novel methods of isolating and differentiating extracellular vesicles (EVs) from a subject, particularly from human induced pluripotent stem cells (hiPSCs). The EVs can be isolated from the spent media of 3D brain organoids differentiated from hiPSCs or from the decellularized extracellular matrix (ECM) of brain organoids.

### CROSS-REFERENCE TO RELATED APPLICATION

This application claims the benefit of U.S. Provisional Application Ser. No. 63/531,823, filed Aug. 10, 2023, the disclosure of which is hereby incorporated by reference in its entirety, including all figures, tables and amino acid or nucleic acid sequences.

### STATEMENT REGARDING FEDERALLY SPONSORED RESEARCH

This invention was made with government support under R01NS125016 awarded by National Institutes of Health. The government has certain rights in the invention.

### BACKGROUND OF THE INVENTION

Extracellular vesicles (EVs) are membrane-bound vesicles that are secreted by all cell types. In particular, EVs secreted by human stem cells participate in intercellular communications and can be used in treating neurological disorders (e.g., immunomodulation, angiogenesis, and neuroprotection). Existing studies have been focusing on generating EVs from tissue or monolayer culture. However, there remains a need for a cell-free based therapy for treating neurological disorders, such as Alzheimer's disease and ischemic stroke.

### BRIEF SUMMARY OF THE INVENTION

The invention pertains to novel methods of isolating and differentiating extracellular vesicles (EVs) from a subject. In certain embodiments, EVs can be isolated from human induced pluripotent stem cells (hiPSCs). In preferred embodiments, EVs can be isolated from the spent media of 3D brain organoids differentiated from hiPSCs, i.e., media EVs (MEVs). In certain embodiments, a novel type of EV subpopulation, matrix-bound nanovesicles (MBVs), can be isolated from decellularized extracellular matrix (ECM) of 3D brain organoids.

In certain embodiments, MEVs and/or MBVs can be harvested from hiPSC-derived forebrain cortical organoids (iFCo). In certain embodiments, MEVs and/or MBVs can be harvested from hiPSC-derived hindbrain cerebellar organoids (iHCo).

In certain embodiments, both MEVs and MBVs have the typical cup shape morphology of exosome, as captured by transmission electron microscopy images. In certain embodiments, the MBVs lack some of detectable EV markers such as, for example, HSC70, CD81, Calnexin, HRS, and syntenin-1; while, MEVs express EV markers, such as, for example, HSC70, CD81, Calnexin, HRS, and syntenin-1, as revealed by a Western blot. In certain embodiments, isogenic microglia and macrophages can be stimulated by Aβ42 oligomers, lipopolysaccharides (LPS), or a combination thereof with the addition of MEVs and MBVs derived from iFCo, as demonstrated by an immunomodulation assay.

### BRIEF DESCRIPTION OF THE DRAWINGS

FIGS. 1A-1C. Schematic of iPSC differentiation and MEV/MBV isolation. (FIG. 1A) iFCo differentiation timeline. (FIG. 1B) iFCo differentiation timeline. (FIG. 1C) iFCo on day 15 and iHCo on day 14 are replated for MEV and MBV isolation. Media of iHCo from week 5 is collected for MEV isolation. For MEV isolation, the conditioned media undergo serial centrifuges. Then mix the media with PEG solution overnight, and finally an ultracentrifuge step is used to collect MEV pallets. For MBV isolation, there is a decellularization step to harvest ECM solutions. It is achieved by the treatment of 0.5% TritonX, then the ECM solutions go through the same process as isolating MEVs.

FIGS. 2A-2C. Characterization of decellularization. (FIG. 2A) Two ECM components—Laminin and Collagen IV-imaged after decellularization. (FIG. 2B) Cells decellularized with Triton X-100 and/or DNAse I. Nuclei are significantly reduced but could not be removed completely. (FIG. 2C) Percentages of remaining DNA of iFCo and iHCo after decellularization. Scale bar: 100 um. B: Before decellularization; T: Triton X-100; T+D: Triton X-100+DNAse I.

FIGS. 3A-3C. Size, yield and purity of iFCo and iHCo MEVs and MBVs. (FIG. 3A) iFCo MBVs were larger than MEVs and their mode sizes were between 100-150 nm. iHCo MEVs and MBVs had size range of 130-140 nm. (FIG. 3B) When normalizing to cell numbers, iFCo and iHCo generated one order magnitude more MBVs than MEVs. (FIG. 3C) When normalizing to proteins, MBVs showed much higher purity than MEVs. n=2-3.

FIGS. 4A-4B. iFCo and iHCo MEVs and MBVs characterized by western blot and TEM. (FIG. 4A) All the EVs show typical exosome markers, while MBVs are absent of or are low at these markers. RCP: iHCo differentiated with RA (retinoic acid), CHIR (CHIR99021), PMR (purmorphamine). CTRL: control. (FIG. 4B) TEM imaging demonstrated that MEVs and MBVs had the typical double layered cup shape. Scale bar for iFCo: 60 um. Scale bar for iHCo: 90 um.

FIGS. 5A-5C. iPSC differentiated isogenic microglia stimulated by Aß42 oligomers with the addition of MEVs and MBVs derived from iFCo MBVs at different doses based on protein content. (FIG. 5A, columns from left to right for each measured parameter: Control, MEV 0.2 (0.2 μg total protein), MEV 1 (1 μg total protein), and MEV 5 (5 μg total protein)). The MEV-0.2 condition showed reduced mRNA expression of the TNFα and IL-6. (FIG. 5B, columns from left to right for each measured parameter: Control, MBV 0.2 (0.2 μg total protein), MBV 1 (1 μg total protein), and MBV 5 (5 μg total protein)) The MBV-1 condition enhanced the expression of IL-10 and CD163. (FIG. 5C) The ELISA analysis of IL-6 protein demonstrated that EV-0.2 condition had the lowest concentration, consistent with the mRNA result. The MBV group showed dose-depended effect on IL-6: the more MBVs added, the less IL-6 was observed.

FIGS. 6A-6C. iHCo MEV biogenesis and iFCo/iHCo MBV miRNA-RT-PCR. (FIG. 6A and FIG. 6B) ESCRT-dependent and independent MEV biogenesis markers were comparable at day 35 iHCo for the two differentiation protocols. (FIG. 6C) 7 out of 8 miRNAs were more abundant in iFCo MBV than iHCo MBV. RCP: iHCo differentiated with RA, CHIR, PMR. CTRL: control.

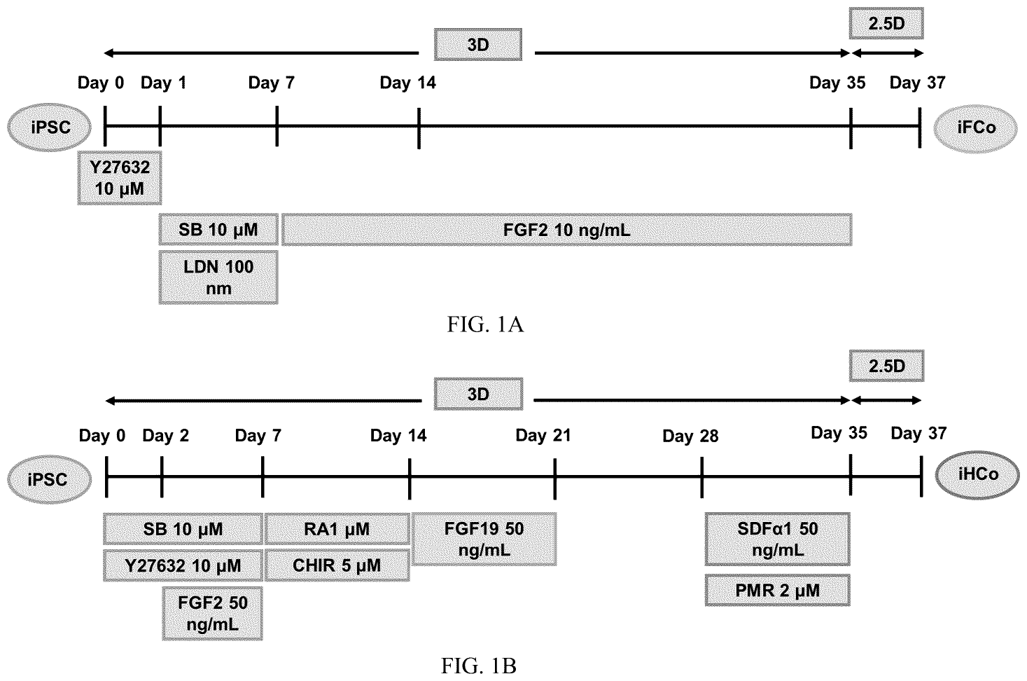

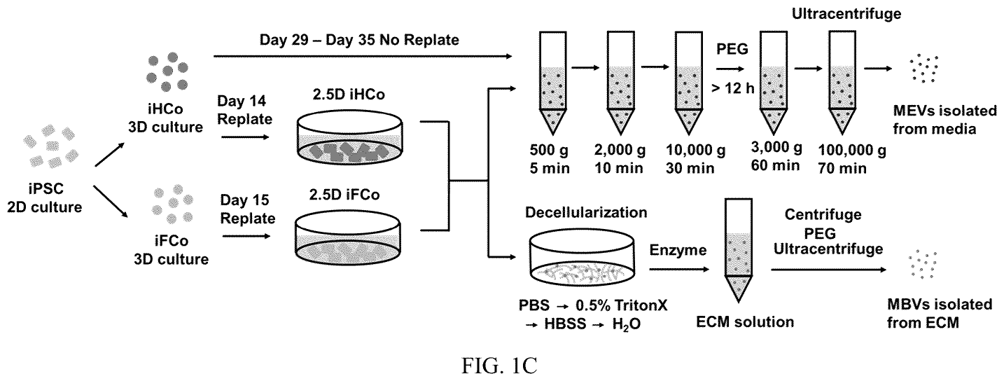

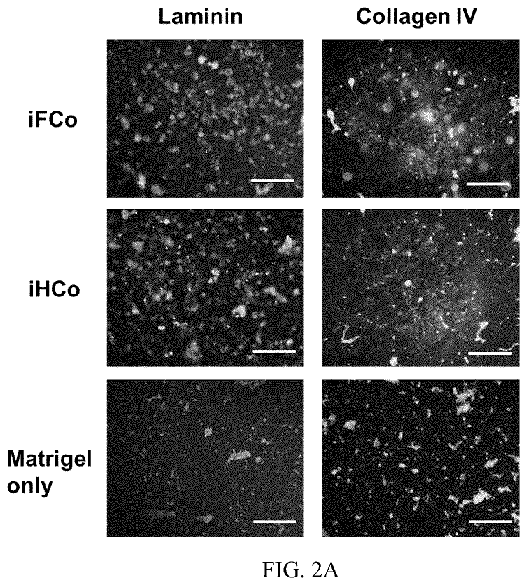

### DETAILED DISCLOSURE OF THE INVENTION

As used herein, the singular forms “a”, “an” and “the” are intended to include the plural forms as well, unless the context clearly indicates otherwise. To the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrase “consisting essentially of” or “consists essentially of” indicates that the described embodiment encompasses embodiments containing the specified materials or steps and those that do not materially affect the basic and novel characteristic(s) of the described embodiment.

The term “about” means within an acceptable error range for the particular value as determined by one of ordinary skill in the art, which will depend in part on how the value is measured or determined, i.e., the limitations of the measurement system. In the context of a period of time in which the term “about” is used, the period of time contains a variation of 0-10% around the value (X±10%). In the context of the term “day”, the term “about” relates to a variance of ±2.4 hours. In the context of compositions containing amounts of ingredients where the terms “about” or “approximately” are used, these compositions contain the stated amount of the ingredient with a variation (error range) of 0-10% around the stated value (X±10%). In the context of pH, the term “about” or “approximately” is intended to include a value of ±0.2 unit of the stated pH.

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values.

As used herein, “subject”, “host” or “organism” refers to any member of the phylum Chordata, more preferably any member of the subphylum vertebrata, or most preferably, any member of the class Mammalia, including, without limitation, humans and other primates, including non-human primates such as rhesus macaques, chimpanzees and other monkey and ape species; livestock, such as cattle, sheep, pigs, goats and horses; domestic mammals, such as dogs and cats; laboratory animals, including rabbits, mice, rats and guinea pigs; birds, including domestic, wild, and game birds, such as chickens, turkeys, ducks, and geese; and reptiles. The term does not denote a particular age or gender. Thus, adult, young, and new-born individuals are intended to be covered as well as male and female subjects. In some embodiments, a host cell is derived from a subject (e.g., tissue specific cells or stem cells). In some embodiments, the subject is a non-human subject. The term “patient” does not necessarily indicate that the subject has been diagnosed with a particular disease, but typically refers to an individual under medical or veterinary supervision. A “patient” can be an individual that is seeking treatment, monitoring, adjustment or modification of an existing therapeutic regimen, etc.

### Methods of Isolating and Differentiating Extracellular Vesicles

The invention pertains to novel methods of isolating and differentiating extracellular vesicles (EVs) from a subject. In certain embodiments, EVs can be isolated from human induced pluripotent stem cells (hiPSCs). In certain embodiments, the hiPSCs can be derived from a fibroblast. In preferred embodiments, EVs can be isolated from the spent media of 3D brain organoids differentiated from hiPSCs, i.e., media EVs (MEVs). In preferred embodiments, a novel type of EV subpopulation, matrix-bound nanovesicles (MBVs), can be isolated from decellularized extracellular matrix (ECM) of 3D brain organoids.

In certain embodiments, the hiPSCs can be differentiated to yield hiPSC-derived forebrain cortical organoids (iFCo) for at least 30, 31, 32, 33, 34, 35, 36, 37, 38, 39, 40, 41, 42, 43, 44, 45, 46, 47, 48, 49, 50, 51, 52, or more days, and then MEVs/MBVs can be harvested on day 1, 2, 3, 4, 5, 6, 7, 8, 9, 10, 11, 12, 13, 14, 15, 16, 17, 18, 19, 20, 21, or 22 after differentiation. In certain embodiments, the MEVs/MBVs, can be harvested on any day of the 30 or more day differentiation period. In certain embodiments, hiPSCs can be incubated with a protein kinase inhibitor, such as, for example, Y-27632 (CAS Registry No. 146986-50-7), at a concentration of about 1 μM to about 20 μM or about 5 μM to about 10 μM for about 1 day. In certain embodiments, hiPSCs can be incubated with an activin receptor-like kinase receptor inhibitor, such as, for example, SB-431542 (CAS Registry No. 301836-41-9) at a concentration of about 1 μM to about 20 μM or about 10 μM, LDN-193189 (CAS Registry No. 1062368-24-4) at a concentration of about 50 nM to about 200 nM or about 100 nM, or a combination thereof for about 6 days to about 8 days, or about 7 days. In certain embodiments, the Y-27632, SB 431542, and/or LDN-193189 can be removed from the incubation by changing the media.

In certain embodiments, hiPSCs can then be incubated with a growth factor protein, such as, for example, Fibroblast growth factor 2 (FGF2), at a concentration of about 10 ng/ml to about 100 ng/ml or about 10 ng/ml for about 7 days to about 35 days. In certain embodiments, the activin receptor-like kinase receptor inhibitor and the protein kinase inhibitor can be removed from the hiPSCs before the addition of the growth factor protein.

In certain embodiments, the resulting iFCo can then be replated to remove the growth factor protein and then harvested about 1.75 days to about 2.25 days or about 2 days after the growth factor protein have been removed. In certain embodiments, the replating process comprises the use of Coat Matrigel (diluted with media at a ratio of 1:50) on attachment wells for at least about 1 h at about 37° C., then removing the Coat Matrigel and seeding cells onto the wells.

In certain embodiments, the hiPSCs can be differentiated to yield hiPSC-derived hindbrain cerebellar organoids (iHCo) for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, about 45, about 50, or more days, and iHCo can be harvested on day 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, about 45, about 50, or preferably on day 14 and during week 5 (e.g., day 29, 30, 31, 32, 33, 34, or 35). In certain embodiments, hiPSCs can be incubated with a protein kinase inhibitor, such as, for example, Y-27632, at a concentration of about 1 μM to about 50 μM or about 10 μM and an activin receptor-like kinase receptor inhibitor, such as, for example, SB-431542 at a concentration of about 1 μM to about 50 μM or about 10 μM for about 6 days to about 8 days or about 7 days. In certain embodiments, hiPSCs can be incubated with a growth factor protein, such as, for example, Fibroblast growth factor 2 (FGF2), at a concentration of about 5 ng/mL to about 100 ng/ml or about 50 ng/ml for about 4 days to about 6 days or about 5 days. In certain embodiments, the incubation of the hiPSCs with the protein kinase inhibitor, the activin receptor-like kinase receptor inhibitor, and the growth factor protein can occur concurrently for about 4 days to about 6 days or about 5 days. In certain embodiments the Y-27632, SB 431542, and FGF2 can be removed from the incubation by changing the media. In preferred embodiments, the growth factor protein can be added to the hiPSCs, the activin receptor-like kinase receptor inhibitor, and the protein kinase inhibitor about 1.75 days to about 2.25 days or about 2 days after the initiation of the incubation of hiPSCs, the activin receptor-like kinase receptor inhibitor, and the protein kinase inhibitor.

In certain embodiments, hiPSCs can then be incubated with a glycogen synthase kinase-3 inhibitor, such as, for example, CHIR99021 (CAS Registry No. 252917-06-9), at a concentration of about 1 μM to about 20 μM or about 5 μM and retinoic acid (RA) at a concentration of about 0.5 μM to about 5 μM or about 1 μM for about 6 days to about 8 days or about 7 days. In certain embodiments, the growth factor protein, activin receptor-like kinase receptor inhibitor, and the protein kinase inhibitor can be removed from the hiPSCs before the addition of the retinoic acid and the glycogen synthase kinase-3 inhibitor.

In certain embodiments, after the hiPSCs have been incubated with retinoic acid and the glycogen synthase kinase-3 inhibitor, the resulting iHCo can be harvested. In certain embodiments, the harvested iHCo can be replated and incubated for about 1.75 days to about 3 days or about 2.5 days after the replating. In certain embodiments, the replating process comprises the use of Coat Matrigel (diluted with media at a ratio of 1:50) on attachment wells for at least about 1 h at about 37° C., then removing the Coat Matrigel and seeding cells onto the wells.

In alternative embodiments, the hiPSCs can be further incubated with a growth factor protein, such as, for example, Fibroblast growth factor 19 (FGF19), at a concentration of about 10 ng/ml to about 100 ng/ml or about 50 ng/mL for about 6 days to about 8 days or about 7 days instead of being harvested and replated. In certain embodiments, the retinoic acid and the glycogen synthase kinase-3 inhibitor can be removed from the hiPSCs before the addition of the growth factor protein.

In certain embodiments, the growth factor protein can be removed from the hiPSCs after the about 7 day incubation, and the hiPSCs can be incubated without any additional compounds for about 6 days to about 8 days or about 7 days.

In certain embodiments, hiPSCs can then be incubated with an alpha chemokine, such as, for example, stromal cell-derived factor 1 (SDF-1) alpha, at a concentration of about 10 ng/ml to about 100 ng/ml or about 50 ng/mL and a hedgehog pathway activator, such as, for example, purmorphamine (PMR), at a concentration of about 0.5 μM to about 5 μM or about 2 μM for about 6 days to about 8 days or about 7 days. In certain embodiments, the iHCo can then be harvested without replating.

In certain embodiments, the iHCo that are harvested without replating can be centrifuged to isolate media extracellular vesicles (MEVs). In certain embodiments, the centrifugation can be a serial centrifugation. In certain embodiments, the serial centrifugation comprises a initial centrifugation follow by at least 1, 2, 3, 4, 5, 6, 7, 8, 9, 10, additional centrifugation steps at a higher force than the previous centrifugation step, a longer centrifugation time than the previous centrifugation step, or a combination thereof. In certain embodiments, the force of the centrifugation is about 500× g to about 100,000 g, about 2,000×g to about 10,000×g, or about 3,000×g. In certain embodiments, the centrifugation time is about 1 min to about 100 min, about 5 mins to about 70 mins, about 10 mins to about 60 mins, or about 30 mins.

In certain embodiments, the iHCo or the iFCo that are harvested with replating can be centrifuged; or, before the centrifugation, the iHCo or iFCo can be decellularized. In certain embodiments, the iHCo or iFCo that are harvested with replating and decellularized can be centrifuged to isolate matrix-bound nanovesicles (MBVs) after decellularization. In certain embodiments, the iHCo or iFCo that are harvested with replating and without decellularization can be centrifuged to isolate MEVs. In certain embodiments, the centrifugation can be a serial centrifugation, as described above.

In certain embodiments, decellularization comprises washing the iHCo or iFCO with a detergent, surfactant, buffer, water or any combination thereof. In preferred embodiments, decellularization comprises washing the iHCo or iFCo with a buffer solution, such as, for example, phosphate buffered saline (PBS), followed by washing with a nonionic surfactant, such as, for example, 0.5% Triton X-100, followed by washing with a balanced salt solution, such as, for example, Hanks' Balanced Salt Solution, and, finally, washing with water. In certain embodiments, after the washing steps, the iHCo or iFCo can be treated with a nuclease, such as, for example DNAse I. In certain embodiments, a washing step is performed by adding washing solutions into wells containing iHCo or iFCo, and then removing the washing solutions.

In certain embodiments, both MEVs and MBVs have the typical cup shape morphology of exosome, as captured by transmission electron microscopy images. In certain embodiments, the MBVs lack some of detectable EV markers such as, for example, HSC70, CD81, Calnexin, HRS, and syntenin-1; while, MEVs express the markers, such as, for example, HSC70, CD81, Calnexin, HRS, and syntenin-1, as revealed by a Western blot. In certain embodiments, isogenic microglia and macrophages can be stimulated by Aß42 oligomers, LPS, or a combination thereof with the addition of MEVs and MBVs derived from iFCo at different doses based on protein content, as demonstrated by an immunomodulation assay.

### Materials and Methods

### Transmission Electron Microscopy (TEM)

Electron microscopy imaging was performed to confirm the morphology and size distribution of EVs as shown previously (Marzano M, Bejoy J, Cheerathodi M R, Sun L, York S B, Zhao J, Kanckiyo T, Bu G, Meckes DG Jr, Li Y. Differential Effects of Extracellular Vesicles of Lineage-Specific Human Pluripotent Stem Cells on the Cellular Behaviors of Isogenic Cortical Spheroids. Cells. 2019 Aug. 28; 8 (9): 993 and Marzano M, Bou-Dargham M J, Cone A S, York S, Helsper S, Grant S C, Meckes D G Jr, Sang Q A, Li Y. Biogenesis of Extracellular Vesicles Produced from Human-Stem-Cell-Derived Cortical Spheroids Exposed to Iron Oxides. ACS Biomater Sci Eng. 2021 Mar. 8; 7 (3): 1111-1122, which are each incorporated by reference it their entireties). Briefly, first, intact EVs (5 μL) were dropped onto Parafilm. A carbon coated 400 Hex Mesh Copper grid (Electron Microscopy Sciences (EMS), Hatfield, PA, USA) was positioned using forceps with coating side down on top of each drop for 1 h. Grids were washed with sterile filtered PBS three times, and then the EV samples were fixed for 10 min in 2% PFA (EMS, EM Grade). After washing, the grids were transferred on top of a 20-μL drop of 2.5% glutaraldehyde (EMS, EM Grade) and incubated for 10 min at room temperature. Grid samples were stained for 10 min with 2% uranyl acetate (EMS, EMS grade). Then the samples were embedded for 10 min with 0.13% methyl cellulose and 0.4% uranyl acetate. The coated side of the grids were left to dry before imaging on the CM120 Biotwin electron microscope (Field Electron and Ion Company, FEI, Hillsboro, OR, USA) (Lässer C, Eldh M, Lötvall J. Isolation and characterization of RNA-containing exosomes. J Vis Exp. 2012 Jan. 9; (59): e3037, which is hereby incorporated by reference in its entirety). Image analysis was performed in ImageJ to determine the average EV size and size distribution.

### Western Blot for EV Markers

EV pellets following ultracentrifugation were lysed in 2× Laemmli sample buffer (4% SDS, 100 mM Tris-HCl [pH 6.8], 0.4-mg/ml bromophenol blue, 20% glycerol) for immunoblot analysis. The supernatant was collected, and a Bradford assay was carried out to determine the protein concentration. Protein lysate concentration was normalized, and 20 μg of each sample was denatured at 95° C. in Laemmli sample buffer with 2-mercaptoethanol. Proteins were separated by 15% Bis-Tris SDS-PAGE gel and transferred onto a nitrocellulose membrane (Bio-Rad, Hercules, CA, USA). The membranes were blocked for 30 min in 5% skim milk power (w/v) in Tris-buffered saline (10 mM Tris-HCl [pH 7.5], and 150 mM NaCl) with 0.1% Tween 20 (v/v) (TBS-T), or in 5% bovine serum albumin in TBS-T. Membranes were incubated overnight in the presence of the primary antibodies (e.g., anti-HRS, anti-Calnexin, anti-HSC70, anti-CD81, and anti-Syntenin-1) diluted in the corresponding blocking buffer at 4° C. Afterward, the membranes were washed four times for 10 min each with TBS-T and then incubated with horseradish peroxidase (HRP)-conjugated secondary antibodies: rabbit anti-mouse IgG (Genetex, Irvine, CA, USA; 26728) or goat anti-rabbit IgG (Fab fragment) (Genetex; 27171) for 1 h at room temperature. After wash with TBS-T, the blots were incubated with SuperSignal West Pico Chemiluminescent Substrate (ThermoFisher Scientific, Waltham, MA, USA; 34080) and imaged using an ImageQuant LAS 4000 (GE Healthcare Bio-Sciences Corp., Piscatoway, NJ, USA) and processed with ImageQuant TL v8.1.0.0 software.

Real-Time Quantitative Reverse Transcription—Polymerase Chain Reactions (qRT-PCR)

For miRNA quantification, total RNA was isolated from different EV and cell samples using the miRNeasy Micro Kit (Qiagen, Valencia, CA, USA) according to the manufacturer's protocol. Reverse transcription was carried out using a commercial qScript miR cDNA synthesis kit (Quantabio, Beverly, MA, USA). The qPCR primer (along with a universal reverse primers: miR-155, miR-221, miR-125b, miR-145, miR-21, miR-22, miR-30b, and miR-133b) for each miRNA has been designed and validated to work specifically with miRNA cDNA reverse transcribed using the kit. The levels of miRNAs were determined, and SNORD44 was used as a housekeeping gene for normalization. qPCR reactions were performed on a QuantStudio 7 Flex Real-time PCR System (Applied Biosystems, Foster City, CA) using SYBR Green PCR Master Mix. The amplification protocol was performed as follows: 10 min at 95° C., and 40 cycles of 95° C. for 15 sec and 60° C. for 30 sec, and 70° C. for 30 sec. Fold variation in gene expression was quantified by means of the comparative Ct method: 2−(ΔCttreatment−ΔCtcontrol), which is based on the comparison of expression of the target gene (normalized to the endogenous control (SNORD44)) between the compared samples.

For mRNA quantification, total RNA was isolated using the RNeasy Plus kit (Qiagen) following vendor's instructions. Reverse transcription was carried out using 2.0 μg of total RNA, anchored oligo-dT primers (Operon) and Superscript III (Invitrogen). Primers for specific target genes, such as, for example, TNFα, IL-6, iNOS, IL-10, CD163, Arginase-1, ALIS, HRS, STAM1, TSG101, CD63, MITF, Rab27b, SMPD2, were designed. qPCR reactions were performed on an ABI7500 instrument (Applied Biosystems) using SYBR Green PCR Master Mix. The amplification reactions were performed, and the quality and primer specificity were verified. Fold variations in gene expressions were quantified using the comparative Ct method: 2−(ΔCttreatment−ΔCtcontrol), which is based on the comparison of the target gene (normalized to endogenous gene (GAPDH)) among different conditions.

### Statistical Analysis

Unless otherwise noted, all experiments were performed at least three times in triplicate (n=3), and the representative data are reported. Experimental results are expressed as means±standard deviation (SD) of the samples. Statistical comparisons were performed by one-way ANOVA and Tukey's post hoc test for multiple comparisons, and significance was accepted at P<0.05.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1—Decellularization

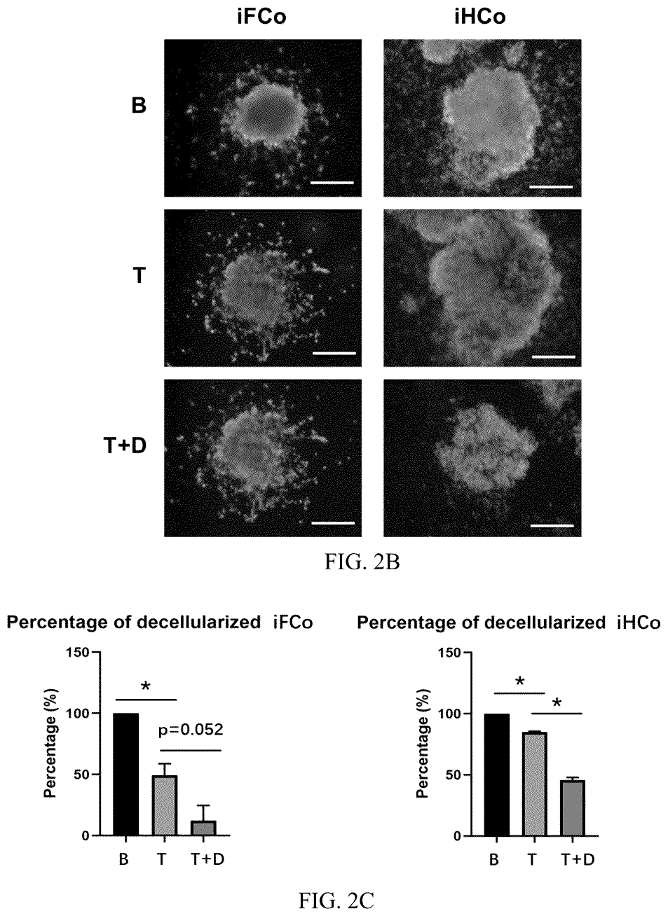

FIG. 2A shows Two ECM components-Laminin and Collagen IV-imaged after decellularization. FIG. 2B shows cells decellularized with Triton X-100 and/or DNAse I. The nuclei are significantly reduced but could not be removed completely. FIG. 2C shows the percentages of remaining DNA of iFCo and iHCo after decellularization in which Triton X-100 decellularization significantly decreased the DNA of iFCo and iHCo and Scale bar: 100 um. B: Before decellularization; T: Triton X-100; T+D: Triton X-100+DNAse I.

### Example 2—Size, Yield, and Purity

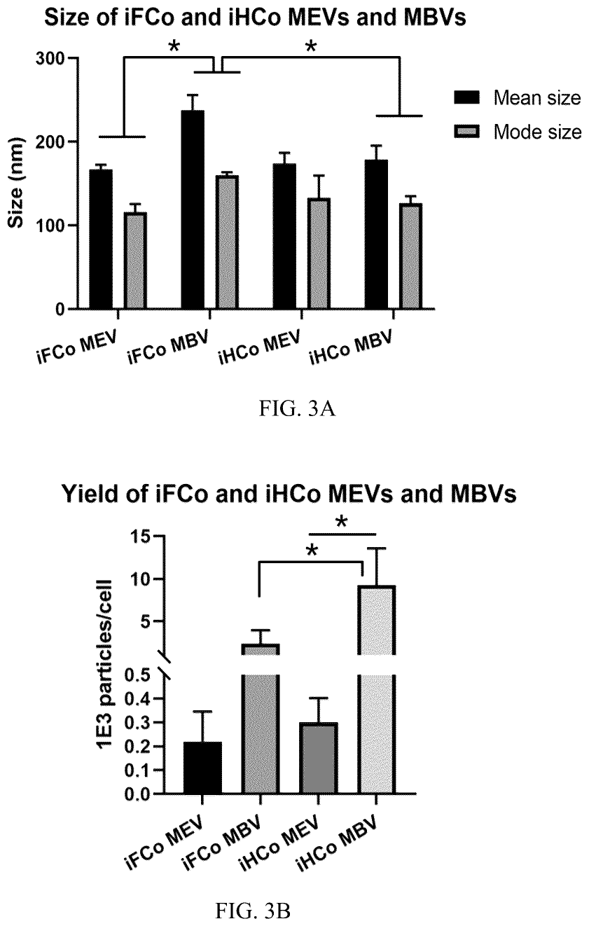

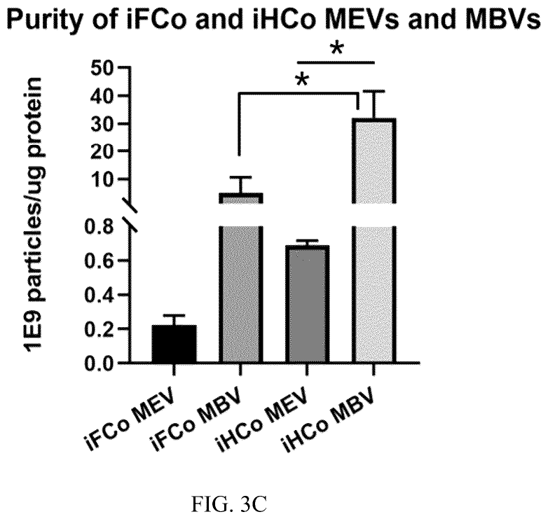

FIG. 3A shows that iFCo MBVs were larger than MEVs, and their mode sizes were between 100-150 nm. iHCo MEVs and MBVs had size range of 130-140 nm. FIG. 3B show that when normalizing to cell numbers, iFCo and iHCo generated one order magnitude more MBVs than MEVs. FIG. 3C shows that when normalizing to proteins, MBVs showed much higher purity than MEVs. n=2-3.

### Example 3—Western Blot and TEM Imaging

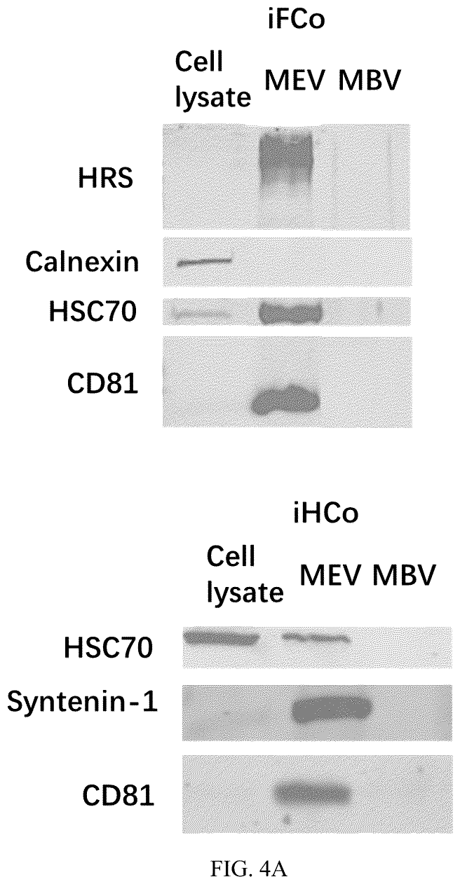

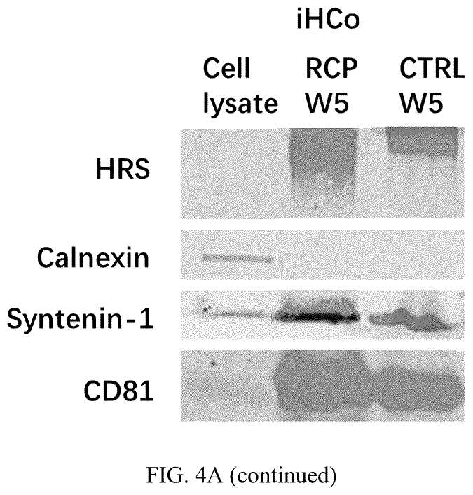

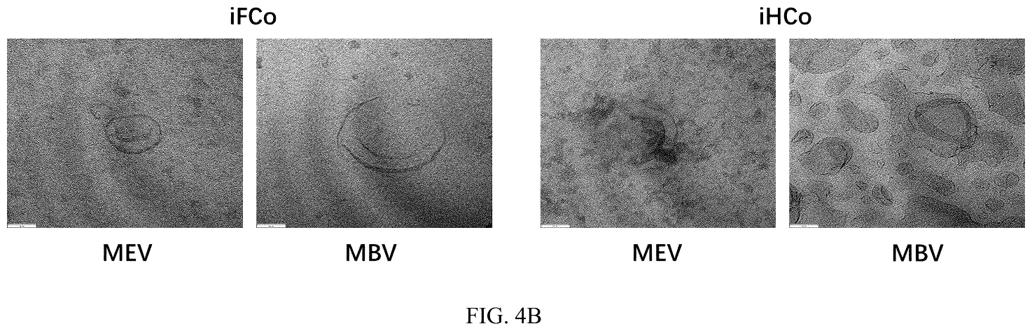

FIG. 4A shows that all the EVs show typical exosome markers, while MBVs are absent of or are low at these markers. RCP: iHCo differentiated with RA, CHIR, PMR. CTRL: control. FIG. 4B shows that TEM imaging demonstrated that MEVs and MBVs had the typical double layered cup shape. Scale bar for iFCo: 60 um. Scale bar for iHCo: 90 um.

### Example 4—Immunomodulation

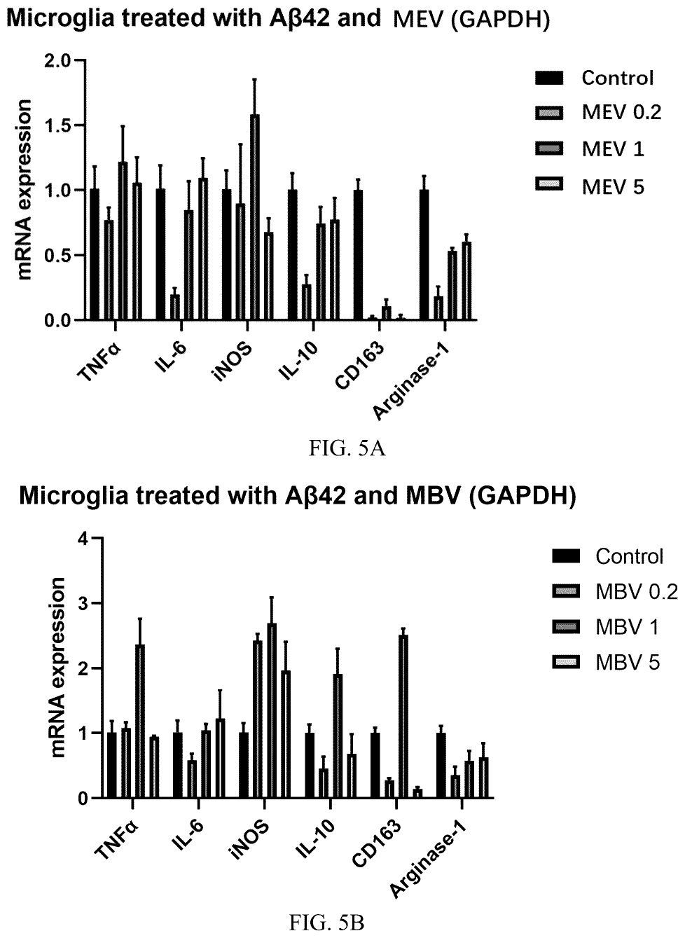

FIG. 5A shows that the MEV-0.2 condition showed reduced mRNA expression of the TNFα and IL-6. FIG. 5B shows that MBV-1 condition enhanced the expression of IL-10 and CD163. FIG. 5C shows that ELISA analysis of IL-6 protein demonstrated that EV-0.2 condition had the lowest concentration, consistent with the mRNA result. The MBV group showed dose-depended effect on IL-6: the more MBVs added, the less IL-6 was observed.

### Example 5—RT-PCR

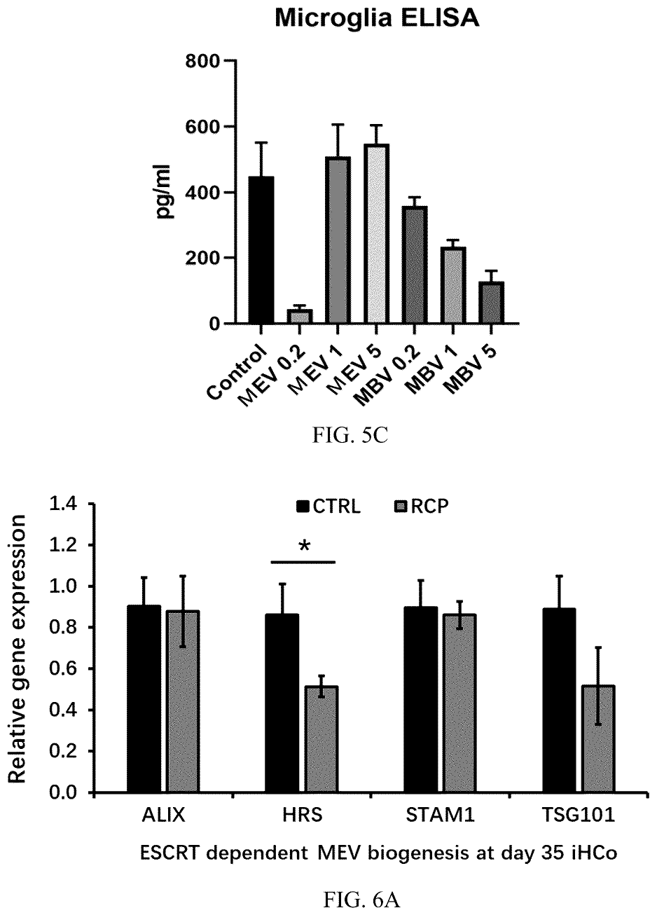

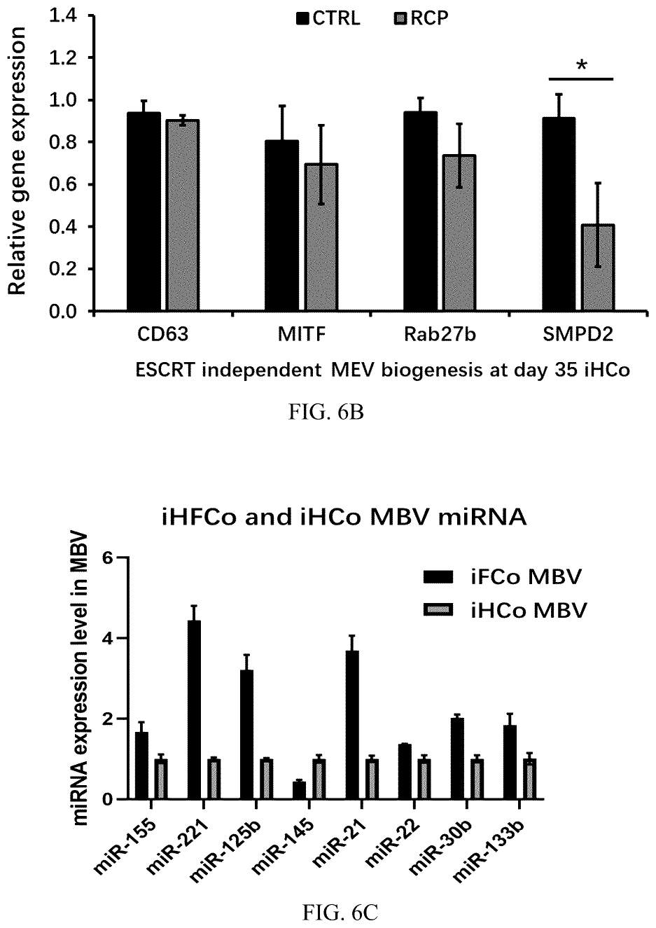

FIG. 6A and FIG. 6B show that ESCRT-dependent and independent MEV biogenesis markers were comparable at day 35 iHCo for the two differentiation protocols. FIG. 6C miRNA cargo analysis shows that 7 out of 8 miRNAs were more abundant in iFCo MBV than iHCo MBV. RCP: iHCo differentiated with RA, CHIR, PMR. CTRL: control.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

### REFERENCES

Yuan X, Sun L, Jeske R, Nkosi D, York S B, Liu Y, Grant S C, Meckes D G Jr, Li Y. Engineering extracellular vesicles by three-dimensional dynamic culture of human mesenchymal stem cells. J Extracell Vesicles. 2022 June; 11 (6): e12235.
Marzano M, Bou-Dargham M J, Cone A S, York S, Helsper S, Grant S C, Meckes D G Jr, Sang Q A, Li Y. Biogenesis of Extracellular Vesicles Produced from Human-Stem-Cell-Derived Cortical Spheroids Exposed to Iron Oxides. ACS Biomater Sci Eng. 2021 Mar. 8; 7 (3): 1111-1122.

We claim:

1. A method for preparing a media extracellular vesicle (MEV) or a matrix-bound nanovesicles (MBV), said method comprising:
i) providing a human induced pluripotent stem cell (hiPSC);
ii) incubating the hiPSC with a protein kinase inhibitor for about 1 day;
iii) incubating the hiPSC with an activin receptor-like kinase receptor inhibitor for about 7 days;
iv) incubating the hiPSC with a growth factor protein for about 7 days to yield a hiPSC-derived forebrain cortical organoid (iFCo);
v) harvesting the iFCo; and
vi) isolating the MEV from the iFCo or isolating the MBV from the iFCo.

2. The method of claim 1, wherein the protein kinase inhibitor is Y-27632.

3. The method of claim 1, wherein the protein kinase inhibitor is at a concentration of about 10 μM.

4. The method of claim 1, wherein the activin receptor-like kinase receptor inhibitor is SB-431542, LDN-193189, or a combination thereof.

5. The method of claim 4, wherein SB-431542 is at a concentration of about 10 μM and LDN-193189 is at a concentration of about 100 nM.

6. The method of claim 1, wherein the growth factor protein is at a concentration of about 10 ng/mL.

7. The method of claim 1, wherein the growth factor protein is Fibroblast growth factor 2 (FGF2).

8. A method for preparing a media extracellular vesicle (MEV), said method comprising:
i) providing a human induced pluripotent stem cell (hiPSC);
ii) incubating the hiPSC with a protein kinase inhibitor and an activin receptor-like kinase receptor inhibitor for about 7 days;
iii) incubating the hiPSC with a first growth factor protein for about 5 days;
iv) incubating the hiPSC with retinoic acid and a glycogen synthase kinase-3 inhibitor for about 7 days;
v) incubating the hiPSC with a second growth factor protein for about 7 days;
vi) removing the second growth factor protein and incubating the hiPSC for about 7 days;
vii) incubating the hiPSC with an alpha chemokine and a hedgehog pathway activator for about 7 days to yield a hiPSC-derived hindbrain cerebellar organoid (iHCo);
viii) harvesting the iHCo; and
ix) isolating the MEV from the iHCo.

9. The method of claim 8, wherein the protein kinase inhibitor is Y-27632.

10. The method of claim 8, wherein the protein kinase inhibitor is at a concentration of about 10 μM.

11. The method of claim 8, wherein the activin receptor-like kinase receptor inhibitor is SB-431542.

12. The method of claim 11, wherein SB-431542 is at a concentration of about 10 μM.

13. The method of claim 8, wherein the first growth factor protein is at a concentration of about 50 ng/mL.

14. A method for preparing a MEV or a matrix-bound nanovesicle MBV, said method comprising:
i) providing a human induced pluripotent stem cell (hiPSC);
ii) incubating the hiPSC with a protein kinase inhibitor and an activin receptor-like kinase receptor inhibitor for about 7 days;
iii) incubating the hiPSC with a growth factor protein for about 5 days;
iv) incubating the hiPSC with retinoic acid and a glycogen synthase kinase-3 inhibitor for about 7 days to yield a hiPSC-derived hindbrain cerebellar organoid (iHCo);
v) harvesting the iHCo; and
vi) isolating the MEV from the iHCo or isolating the MBV from the iHCo.

15. The method of claim 14, wherein the protein kinase inhibitor is Y-27632.

16. The method of claim 14, wherein the protein kinase inhibitor is at a concentration of about 10 μM.

17. The method of claim 14, wherein the activin receptor-like kinase receptor inhibitor is SB-431542.

18. The method of claim 17, wherein SB-431542 is at a concentration of about 10 μM.

19. The method of claim 14, wherein the growth factor protein is at a concentration of about 50 ng/mL.
